(19) United States
(12) Patent Application Publication
(10) Pub. No.: US 2004/0132821 A1
(43) Pub. Date: Jul. 8, 2004

(54) Therapeutic combination of carnitine and antioxidant polyphenols

(76) Inventor: Roberto Crea, San Mateo, CA (US)

Correspondence Address: QUARLES & BRADY LLP, RENAISSANCE ONE, TWO NORTH CENTRAL AVENUE, PHOENIX, AZ, 85004-2391, US

(21) Appl. No.: 10/683,803

(22) Filed: Oct. 10, 2003

Related U.S. Application Data
(60) Provisional application No. 60/417,838, filed on Oct. 11, 2002.

(51) Int. Cl. A61K 31/70; A61K 31/205; A61K 31/198

(52) U.S. Cl. 514/563; 514/23; 514/554

(57) ABSTRACT

A composition includes acetyl-L-carnitine or its pharmacologically acceptable salt and a mixture of polyphenols containing hydroxytyrosol in an effective weight ratio. Carnitine can be L-carnitine, propionyl-L-carnitine, valeryl-L-carnitine, isovaleryl-L-carnitine or mixtures thereof. The composition has a weight ratio of carnitine:hydroxytyrosol of from 100:1 to 1:10. Also disclosed is a method of preventing tissue damage caused by the presence of free radicals due to environmental pollution; of preventing brain or myocardial lesions induced by free radicals following cerebral or myocardial ischemia and reperfusion; of preventing diabetic or toxic neuropathies and metabolic disorders of glucose utilization, which includes administering the composition.

This application claims the benefit of U.S. Provisional Application No. 60/417,838, filed Oct. 11, 2002.

### BACKGROUND

1. Technical Field

The present invention relates to a composition for the prevention and/or treatment of diseases due to the presence of free radicals, and more specifically to a combination of carnitine with polyphenols, including hydroxytyrosol.

2. The Prior Art

A systemic deficiency of alkanoyl-L-carnitines (ubiquitous, naturally occurring compounds, the greatest concentrations of which are in skeletal muscle and myocardium) leads to muscular and functional deficits, which can be restored to normal by the administration of these compounds.

Acetyl-L-carnitine is observed in the brain and in peripheral nervous tissue, where its presence is necessary, for normal nerve conduction. The production of energy by carnitines occurs via intra-mitochondrial β-oxidation of fatty acids, the oxidation of branched-chain amino acids, and regulation of insulin activity. Important studies of carnitines indicate their stabilizing effects on cellular phospholipid membranes and on the integrity and deformability of erythrocytes.

Acetyl-L-carnitine protects cerebral tissue against oxidative phenomena. Carnitine is necessary for normal growth. Reduced carnitine levels have been detected during aging. During the metabolic processes associated with aging, increased oxidative processes are detected together with a related increase in free radicals, the presence of which mediates the onset of diabetic lesions.

Impaired mitochondrial activity leads to an increase in oxidants, which the cell defenses are no longer able to combat effectively. The increase in peroxides, hydroxides and singlet oxygen produced by aerobic metabolism leads to damage to macromolecules (DNA, proteins and lipids), which in turn contributes to the onset of degenerative diseases, including diabetes. The reduced mitochondrial activity which comes with aging is also accompanied by a reduction in cardiolipin, a diphosphatidyl-glycerol derivative which is part of the structure of the mitochondrial membrane and plays an important role in maintaining mitochondrial activity, particularly at the level of fatty acid β-oxidation processes. Mitochondrial activity, including the fatty acid β-oxidation processes, can be enhanced by the administration of acetyl-L-carnitine, which can restore normal cardiolipin concentrations in the mitochondria.

The positive effect of acetyl-L-carnitine on mitochondrial activity is also proved reflected in improved utilization of the glycolytic pathway for ATP production.

Recently, antioxidants have been shown to regulate glucose utilization and insulin activity. Lipid peroxidation, which is increased in diabetic neuropathy, can be controlled and reduced, both at cerebral level and at the level of the sciatic nerve and the ocular lens, by the administration of antioxidants. Moreover, antioxidants inhibit the aldose reductase activated by hyperglycemia. Therefore, antioxidants may be an important in diabetic therapy. An antioxidant effect has been shown to protect against brain damage induced by ischemia and has a postulated therapeutic role in Parkinson's disease and AIDS.

Antioxidants may work either directly or indirectly, via restoration of glutathione and ascorbic acid concentrations. For example, α-lipoic acid directly affects carbohydrate metabolism by acting as a coenzyme in the oxidative decarboxylation of pyruvate and other α-ketoacids. It acts indirectly through the acetates, in the tricarboxylic acid cycle leading to the formation of ATP.

Because many of the complications associated with diabetes, such as neuropathies and ocular cataracts, are mediated by reactive oxygen species (ROS), inhibition of activation of the nuclear transcription factor is another possible mechanism by which an antioxidant may prevent diseases related to diabetes. Furthermore, in diabetic subjects, the concentrations of α-lipoic acid are lower than normal values, and the administration of α-lipoic acid may restore these levels to normal. It has an additive effect to that of insulin in glucose transport to the cell membranes.

Chronic exposure to high concentrations of glucose may lead to a reaction between glucose and proteins and to the spontaneous formation of highly reactive proteins known as end products of glycosylation (Advanced Glycosylation End products or AGEs). Important among these are the glycosylation products of glucose and albumin, glucose and collagen, and glucose and hemoglobin. AGEs affect tissues and cells in a large proportion of diabetic diseases at nervous, muscular and endothelial level. In fact, AGEs enhance the synthesis of the components of the extracellular matrix; increase endothelial permeability and the formation of immune complexes and cytokines; and cause neuronal and retinal ischemia, myelin accumulation and myelin degeneration. A number of these compounds are formed in both diabetes and aging.

A correlation between AGEs and activation of NF-IKB has recently been demonstrated, as has the ability of α-lipoic acid to inhibit this reaction. Protein glycation and glucose oxidation at high concentrations together with free radicals may therefore cause tissue abnormalities—particularly nerve tissue abnormalities—associated with diabetes. α-Lipoic acid also inhibits or limits glycosylation or glucose oxidation reactions.

Another protective effect of α-lipoic acid has also been observed in pancreatic cells placed in contact with inflammatory agents. In addition to sparing vitamin E and increasing glutathione concentrations, the protective action of antioxidants against the onset of neuropathies has also been confirmed. The reduction in nervous lesions is also accompanied by a reduction in oxidative reaction products, especially malonyl aldehyde concentrations. Other studies have confirmed antioxidant activity in the treatment of diabetic neuropathies.

### SUMMARY OF THE INVENTION

In one embodiment, a composition comprises acetyl-L-carnitine, or a pharmacologically acceptable salt thereof, and a mixture of polyphenols containing hydroxytyrosol in an effective weight ratio. The carnitine is selected from the group consisting of L-carnitine, propionyl-L-carnitine, valeryl-L-carnitine, isovaleryl-L-carnitine and their pharmacologically acceptable salts or mixtures thereof. The composition has a weight ratio of carnitine:hydroxytyrosol which is from 100:1 to 1:10. Pharmacologically acceptable salts of acetyl-L-carnitine or alkanoyl-L-carnitine are selected from the group consisting of chloride, bromide, iodide, aspartate, acid aspartate, citrate, acid citrate, tartrate, phosphate, acid phosphate, fumarate, acid fumarate, glycerophosphate, glucose phosphate, lactate, maleate, acid maleate, orotate, acid oxalate, sulphate, acid sulphate, trichloroacetate, trifluoroacetate and methane sulfonate. The composition further comprises vitamins, coenzymes, mineral substances or other antioxidants. The composition can be an orally administered form, such as a dietary supplement. The composition can be orally, parenterally, rectally or transdermally administered as a medicament. The composition can be in solid, semi-solid or liquid form. The composition can be in the form of tablets, lozenges, pills, capsules, granulates, syrups, injection or drops.

Another embodiment of the invention is a method of preventing tissue damage brought about by the presence of free radicals due to environmental pollution, for preventing brain or myocardial lesions induced by free radicals following cerebral or myocardial ischemia and attendant reperfusion, for preventing diabetic or toxic neuropathies, or for metabolic disorders in glucose utilization. The method comprises administering to a subject in need of same a composition which comprises acetyl-L-carnitine or a pharmacologically acceptable salt thereof and also a carnitine selected from the group consisting of L-carnitine, propionyl-L-carnitine, valeryl-L-carnitine, isovaleryl-L-carnitine or their pharmacologically acceptable salts or mixtures thereof; and a mixture of polyphenols containing hydroxytyrosol.

Another embodiment is a method of treating a disease brought about by the presence of free radicals due to environmental pollution, brain or myocardial lesions induced by free radicals following cerebral or myocardial ischermia and attendant reperfusion, atherosclerosis lesions and tissue proliferative processes, diabetic or toxic neuropathies, and of metabolic disorders in glucose utilization. This method comprises administering to a subject in need of same a composition comprising acetyl-L-carnitine, or a pharmacologically acceptable salt thereof, and optionally also a carnitine selected from the group consisting of L-carnitine, propionyl L-carnitine, valeryl L-carnitine, isovaleryl L-carnitine or their pharmacologically acceptable salts or mixtures thereof; and a mixture of polyphenols containing hydroxytyrosol. Carnitine and hydroxytyrosol are administered in a weight ratio of from 100:1 to 1:10.

### DETAILED DESCRIPTION OF A PREFERRED EMBODIMENT

Natural antioxidants present in high concentration in olives continue to capture the interest of medical science. There is a growing body of evidence documenting the unique antioxidant activity of a family of compounds, polyphenols, against oxygen-derived free radicals in pathological processes. The health benefits of extra virgin olive oil in the promotion of healthy breast tissue, colon function, cardiovascular function and other health states associated with oxidative stress can be attributed to the strong antioxidant activity of polyphenols. These antioxidants may also help maintain overall health and wellness through their antibacterial and antiviral activity.

Specifically oleuropein and hydroxytyrosol are the natural polyphenols from olives that provide the highest level of free radical protection ever reported for any natural antioxidant compound. Hydroxytyrosol is also known as 3,4-dihydroxyphenylalanine. D'Angelo, et al. (DMD 29:1492-1498, 2001), determined that hydroxytyrosol is the major component of olive oil fractions. Phenolic compounds, including hydroxytyrosol, are known to have powerful antioxidant properties.

The antioxidant action of hydroxytyrosol is supported by observations of Visioli, et al. (Circulation 102:2169-71, 2000), who found that F2-isoprostanes (a biomarker of oxidative status, such as lipid peroxidation) are suppressed in animals exposed to the pro-oxidant effects of passive smoking. In normal humans he found a dose-dependent decrease in urinary F2-isoprostanes (8-epi-PGFs, or 8-epi), and a negative correlation between 8-epi and homovanillyl alcohol (HVA1c), the major metabolite of hydroxytyrosol (Visioli et al., Biochem Biophys Res Commun 278:797-799, 2000). Catechol-O-methyl transferase (COMT) enzymes involved in catecholamine catabolism act on hydroxytyrosol, resulting in an enhanced excretion of HVA1c (Caruso, et al., Metabolism 50:1426-28, 2001). Further research has proven that hydroxytyrosol has the ability to scavenge peroxynitrite, which is both a reactive nitrogen species (RNS) and a reactive oxygen species (ROS) (de la Puerta, et al., Life Sci 69:1213-22, 2001).

After ingestion humans dose-dependently absorb hydroxytyrosol. The preferential route of excretion is as glucuronide conjugates in urine (D'Angelo et al., DMD 29:1492-98, 2001). Bonanome et al. (Nutr Metab Cardiovasc Dis 10:111-120, 2000) suggest that peak postprandial concentrations of hydroxytyrosol in olive oil occur 1-2 hours after consumption of a meal corresponding to peak antioxidant activity (TAC). Such phenols are rapidly cleared from the plasma and are not observed in the fasting state. When administered intravenously and orally in an olive oil solution and dosed as an aqueous solution, oral bioavailability was 99% and 71%, respectively (Tuck et al., J Nutr 131:1993-96, 2001).

Hydroxytyrosol is considered a simple, lipophilic compound. However, its antioxidant properties are more potent than vitamin E and reveal strong metal chelation and free radical scavenging action similar to α-lipoic acid. Of considerable importance, hydroxytyrosol scavenges superoxide anions, unlike α-lipoic acid, which does not. Visioli and Galli (Curr Atheroscl Rep 3:64-67, 2001) listed some of the biological activities of olive oil polyphenols as a) inhibition of low density lipoprotein oxidation, b) inhibition of platelet aggregation, c) scavenging of superoxide and other ROS, d) inhibition of peroxynitrite-induced DNA damage and tyrosine nitration, e) increased nitric oxide production by lipase-challenged macrophages, f) hypotensive action and g) increased TAC.

There is a proprietary two-step process to utilize olive water rich in harvesting antioxidant polyphenols. First, the pits are removed from the olives. Next the pitted olives are pressed, which removes both water and oil from the olives. Then the polyphenolic-rich water is separated from the oil and processed to avoid air oxidation and to release the highest quantity of antioxidant activity. Olive water from the olive oil milling process, or vegetation water, thus becomes an economical and yet previously unused source of natural antioxidant polyphenols (including hydroxytyrosol). Two patents, U.S. Pat. Nos. 6,165,475 and 6,197,308, both to Roberto Crea, describe this process in detail and are hereby incorporated by reference. Oleuropein also is commercially obtained from olive leaves and can be added to the aqueous extract of olives.

The vegetation water can be acidified to between pH of 2.0 and 4.0 to convert oleuropein to hydroxytyrosol (U.S. Pat. No. 6,165,475). U.S. patent application Ser. No. 09/944,744, hereby incorporated by reference, discloses additional ways to purify for hydroxytyrosol, including aqueous-alcoholic extraction. The weight ratio of hydroxytyrosol to oleuropein is preferably between 1:1 and 400:1, more preferably between about 3:1 and about 200:1 and most preferably between about 5:1 and 100:1. The extracts may also be formulated to contain various weight ratios of hydroxytyrosol and tyrosol of between about 10:1 and about 50:1, and preferably between about 15:1 and about 30:1.

The polyphenols are provided as about 0.05-20 mg/kg hydroxytyrosol in about 1-200 mg/kg polyphenols. Preferably there is about 0.15-10 mg/kg hydroxytyrosol in 10-100 mg/kg polyphenols. More preferably there is about 0.2-5 mg/kg hydroxytyrosol in about 15-50 mg/kg polyphenols. Most preferably, there is about 0.3-0.4 mg/kg hydroxytyrosol in about 15-25 mg/kg polyphenols.

The polyphenols and carnitine(s) can be administered orally or parenterally. Oral dosage forms can be in a solid or liquid form. Such dosage forms can be formulated from purified polyphenols, or they can be formulated from aqueous or aqueous-alcoholic extracts. Regarding the latter, aqueous or aqueous alcoholic (e.g., water-methanol or water-ethanol) extracts can be spray-dried to provide a dry powder that can be formulated into oral dosage forms with other pharmaceutically acceptable carriers.

The solid oral dosage form compositions are prepared in a manner well known in the pharmaceutical arts, and comprise polyphenols and carnitine(s) in combination with at least one pharmaceutically acceptable carrier. In making such compositions, the polyphenols (either in substantially pure form or as a component of a raw distillate or extract) are usually mixed, diluted or enclosed with a carrier. The carrier can be in solid form, semi-solid or liquid material that acts as a vehicle, carrier or medium for the active ingredient. Alternatively, the carrier can be in the form of a capsule or other container to facilitate oral administration. Thus, the solid oral dosage forms for administration in accordance with the present invention can be in the form of tablets, pills, powders, or soft or hard gelatin capsules.

Polyphenols and carnitine(s) can be formulated with other common pharmaceutically acceptable excipients, including lactose, dextrose, sucrose, sorbitol, mannitol, starches, gums, calcium silicate, microcrystalline cellulose, polyvinylpyrrolidone, methyl cellulose, water, alcohol and the like. The formulations can additionally include lubricating agents such as talc, magnesium stearate and mineral oil, wetting agents, emulsifying and suspending agents, preserving agents such as methyl- and propylhydroxybenzoates, sweetening agents or flavoring agents. Further, the polyphenols and/or carnitine(s) can be formulated to provide quick, sustained or delayed release of the active ingredient after administration to a subject.

Alternatively, the polyphenols and/or carnitine(s) can be in liquid form wherein the pharmaceutically acceptable carrier is water or an aqueous-alcoholic (e.g., ethanol) medium. Parenteral formulations of polyphenols and carnitines are prepared using standard techniques in the art. They are commonly prepared as sterile injectable solutions, using a parenterally acceptable carrier such as isotonic saline prior to administration to a subject.

What is meant by pharmacologically acceptable salt of L-carnitine or alkanoyl-L-carnitine is any salt of these active ingredients with an acid that does not give rise to unwanted toxic or side effects. Non-limiting examples of suitable salts are the following: chloride; bromide; iodide; aspartate, acid aspartate; citrate, acid citrate; tartrate; phosphate, acid phosphate; fumarate, acid fumarate; glycerophosphate, glucose phosphate; lactate; maleate, acid maleate; orotate; oxalate, acid oxalate; sulphate, acid sulphate, trichloroacetate, trifluoroacetate and methane sulphonate. A list of FDA-approved pharmacologically acceptable salts is given in Int J of Pharm, 33:(1986), 201-217; this publication is, incorporated herein by reference.

The composition according to the invention may also comprise vitamins, coenzymes, minerals substances and antioxidants.

Appropriate excipients to be used to prepare the compositions having regard to the specific route of administration, will be apparent to the pharmacy and food industry experts.

### EXAMPLE 1

### Toxicological Tests

Both carnitines and hydroxytyrosol are well known for their low toxicity and good tolerability. These favorable toxicological characteristics of carnitines and hydroxytyrosol have been confirmed. In rats and mice, in fact, it proved possible to administer amounts of up to 250 mg/kg of acetyl-L-carnitine or 1 g /kg of polyphenols including hydroxytyrosol parenterally.

Safety and toxicity studies at 0.5, 1 and 2 g/kg of hydroxytyrosol and other phenolic compounds indicated no clinical or pathological toxicity in mice (Primeca Redfield Study). McGuire (personal communication), studying a polyphenol mixture among a population with HIV-Associated Dementia or Minor Cognitive-Motor Syndrome, administered daily 20 mg of hydroxytyrosol within 1200 mg of polyphenols and reported no adverse events during a six-week study.

Also prolonged administration via the diet for 30 consecutive days, both in rats, and mice, of 200 mg/kg of acetyl-L-carnitine or 200 mg/kg of the carnitine mixture together with 100 mg/kg of hydroxytyrosol was tested, with the results that it was well tolerated and led to no detection of signs of toxicity. Both the weight gain and the various blood-chemistry tests performed in these animals were determined, as were the findings of histopathology tests performed on the main organs after sacrificing the animals at the end of treatment.

### EXAMPLE 2

### Neuroprotective Activity Tests in Experimental Cerebral Ischemia

Lesions due to cerebral ischemia are related to the production of free radicals and of nitrous oxide, and both carnitines and antioxidants afford protection against the toxic action of free radicals. In these tests cerebral ischemia is induced by occluding the middle cerebral artery (MCA) according to the method described by Scharkey (Scharkey, Y., Nature 371-336, 1994) by injecting endothelin-1 (120 pmol in 3 nl) into the anesthetized rat over three minutes with a microcannula placed stereotactically in the piriform cortex at the level of the MCA. Occlusion of the MCA is induced, and the resulting ischemia is to be checked three days after this procedure after sacrificing the rats with transcardiac perfusion of a solution of paraformaldehyde (4% in PBS).

After removal, the brain is placed in fixative containing 10% sucrose, and cryostatic sections (20 nm) fixed with cresyl violet are examined under the optical microscope. Acetyl-L-carnitine (50 mg/kg), or the carnitine mixture (50 mg/kg of a mixture of acetyl-L-carnitine, propionyl-L-carnitine, and isovaleryl-L-carnitine in a 1:1 weight ratio to one another), or hydroxytyrosol (20 mg/kg) (Table 1) are administered intravenously 5 minutes after the endothelin injection.

TABLE 1
Treatment groups

Acetyl-L-carnitine
Hydroxytyrosol
Carnitine mixture
Acetyl L-carnitine + hydroxytyrosol
Carnitine mixture + hydroxytyrosol

The volume of the infarcted area is calculated according to the method described by Park (Park, C., Ann Neurol, 24:543-51, 1988). The results of these tests are intended to determine whether acetyl-L-carnitine, the carnitine mixture, and hydroxytyrosol are capable of reducing the ischemic area; but it is expected that the greatest and most significant result would occur with a combination of these products and, in particular, with a combination of acetyl L-carnitine and hydroxytyrosol.

### EXAMPLE 3

### Experimental Diabetic Hyperglycemia Tests

Controlling serum glucose is one of the most important means of preventing diseases related to diabetes. In these tests, experimental diabetes is induced in rats; and tests are then performed to establish whether the induced hyperglycemia could be reduced by the administration of acetyl L-carnitine, or carnitine mixture, or hydroxytyrosol, or combinations of these products (Table 2). The hyperglycemia is induced by subcutaneous injection of alloxan (100 mg/kg) in the rat, and those rats are considered hyperglycemic that presented serum glucose levels above 450 mg/dl seven days after the alloxan injection.

Treatment with the test substances is given orally for a period of three weeks. At the end of this period, serum glucose is measured in the various groups of rats, both hyperglycemic and treated.

The results obtained are intended to show whether both carnitines and hydroxytyrosol alone or in combination are capable of lowering the high initial serum glucose values.

TABLE 2
Treatment Groups

Controls
Acetyl-L-carnitine
Carnitine mixture
Hydroxytyrosol 4 mg/kg
Acetyl L-carnitine + hydroxytyrosol
Carnitine mixture + hydroxytyrosol
Acetyl L-carnitine = 200 mg/kg
Carnitine mixture = acetyl-L-carnitine +
propionyl-L-carnitine + isovaleryl-
L-carnitine in a 1:1 ratio to one another
Hydroxytyrosol = 4 mg/kg

### EXAMPLE 4

### Tests of Sorbitol Content in Ocular Lens and Sciatic Nerve of the Diabetic Rat

One of the most frequent causes of lesions induced by diabetic hyperglycemia is the intracellular accumulation of sorbitol, with consequent reduction of osmotic capacity and cell integrity.

This has been attributed as a cause of the ocular and peripheral nerve conditions. Tests are conducted in a group of rats in which diabetes is induced, for example, by means of the intravenous administration of 50 mg/kg of streptozotocin. One week after injection, serum glucose is tested; and those rats are considered diabetic with serum glucose values above 450 mg/dl. The diabetic rats then receive intraperitoneal injections for eight consecutive days of the following: acetyl-L-carnitine (100 mg/kg); or carnitine mixture (acetyl-L-carnitine+propionyl-L-carnitine+isovaleryl-L-carnitine in a 1:1 weight ratio to one another) (100 mg/kg); or hydroxytyrosol (5 mg/kg), either alone or in various combinations (Table 3).

Before and after eight days of treatment, after suitable isolation, the sorbitol concentration in the sciatic nerve and the ocular lens of diabetic rats is measured. A decrease in the sorbitol concentration is interpreted as less nerve and lens diabetic damage.

TABLE 3
Treatment Groups

Diabetics + Acetyl-L-carnitine
Carnitine mixture
Hydroxytyrosol
Acetyl-L-carnitine + Hydroxytyrosol
Carnitine mixture + Hydroxytyrosol

### EXAMPLE 5

### Sciatic Nerve Regeneration Tests in Diabetic Rats

Rats with induced diabetes whose sciatic nerve has been cut are known to present inferior regenerative activity to that of normal rats. These tests are conducted to investigate whether regeneration of the sciatic nerve in diabetic rats may be accelerated by treatment with acetyl-L-carnitine, carnitine mixture, or hydroxytyrosol, or combinations of these products. The technique used in these tests is the one described by Fernandez (Fernandez, E., Int J Clin Pharmacol Res, 10:85, 1990).

Diabetes (serum glucose above 450 mg/dl) is induced in a group of rats by subcutaneous injection of 100 mg/kg of alloxan. Acetyl-L-carnitine, carnitine mixture and hydroxytyrosol are administered with the diet in such a way that the daily intake was 200 mg/kg of acetyl-L-carnitine, 200 mg/kg of carnitine mixture (acetyl-L-carnitine+propionyl-L-carnitine+isovaleryl-L-carnitine in a 1:1 weight ratio to one another) and 5 mg/kg of hydroxytyrosol (Table 4). The compounds are administered a week before cutting the sciatic nerve and for thirty days after cutting.

The sciatic nerve is cut under anesthesia and after exposing 1 cm of it at the level of the sciatic foramen. The border of the lesion is marked with an epineural suture. Thirty days after cutting the nerve, the tissue of the tibial nerve, one of the main divisions of the sciatic nerve, is examined, after sacrificing the animals. Four cross-sections of the tibial nerve measuring approximately 4 mm in length are thus subjected to morphological and morphometric examination by means of a semiautomatic image analyzer (such as the Zeiss Videoplan Image Analyser).

The number of regenerating axons and their density per 100 nm² are counted, as well as the degenerate elements. This is used to assess the diabetes-induced degeneration of the tibial nerve elements, which may be corrected by treatment with acetyl-L-carnitine, carnitine mixture, and hydroxytyrosol.

TABLE 4
Treatment

Controls
Acetyl-L-carnitine
Carnitine mixture
Hydroxytyrosol
Acetyl-L-carnitine + hydroxytyrosol
Carnitine mixture + hydroxytyrosol

### EXAMPLE 6

### Neuromuscular Conduction Tests

One of the most evident abnormalities in peripheral neuropathies and particularly in diabetic neuropathy is the slowing of neuromuscular conduction, which is reflected in changes in motor activity. In these tests, we induce experimental diabetes in rats by intravenously injecting control and experimental animals (rats with a mean weight of 300 g) with 50 mg/kg of streptozotocin. The experimental animals are treated as shown in Table 5. In the animals with induced diabetes (serum glucose above 450 mg/dl), the neuromuscular conduction velocity (NMCV) is measured. To this end, the sciatic nerve is isolated (2 cm length). The soleus muscle is separated from the gastrocnemius and its distal tendon cut and connected to an isometric transducer which records the muscular contraction force (MCF). The muscle is stimulated via the sciatic nerve by means of two electrodes inserted at a distance of 10 mm from the nerve and connected to a stimulator. A bipolar electrode is placed at the distal end of the gastrocnemius. The electromyogram is displayed on an oscilloscope. The NMCV is measured in m/sec and derives from dividing the distance between the stimulation electrodes by the mean difference in latency between the start of the ECG potentials evoked in the two sites. The MCF is expressed in mm.

TABLE 5
Treatment Groups

Controls
Diabetics
Diabetics + acetyl L-carnitine
Diabetics + carnitine mixture
Diabetics + hydroxytyrosol
Diabetics + acetyl L-carnitine + hydroxytyrosol
Diabetics + carnitine mixture + hydroxytyrosol

### EXAMPLE 7

### Motor Co-ordination Abnormality Test

These tests are conducted in “wobbler mice”, which present an unsteady, staggering gait; an abnormal position of the paws; and a reduced speed of movement. These abnormalities are due to progressive atrophy of the motor neurons and musculo-cutaneous nerve fibers, particularly those affecting the anterior limbs. Tests are conducted according to the procedure proposed by Mitsumoto (Mitsumoto, H., Ann Neurol 36:142-8, 1994). After diagnosis the wobbler mice are treated orally for twenty days consecutively with acetyl-L-carnitine (200 mg/kg), or with carnitine mixture (200 mg/kg), or with hydroxytyrosol (5 mg/kg), or with these products in various combinations (Table 6). The examination is performed by evaluating, in the treated animals versus controls, the time that each animal holds onto the edge of an inclined platform (holding time) and also the time it takes to run a distance of 10 cm (running time).

TABLE 6
Treatment groups

Controls
Acetyl-L-carnitine
Carnitine mixture
Hydroxytyrosol
Acetyl-L-carnitine + hydroxytyrosol
Carnitine mixture + hydroxytyrosol

On the basis of the synergistic interaction of its components, the composition according to the invention described herein is suitable for preventing toxic and metabolic damage which gives rise to neuronal lesions of an acute or chronic nature. In particular, it can be used in the treatment of toxic neuropathies, especially diabetic peripheral neuropathies.

In view of its antioxidant capability, this composition is also indicated in the prevention or treatment of abnormalities of toxic or anoxic nature and related to the release of free radicals in the brain, liver, heart or other organs and tissues.

Furthermore, in view of the ability of the composition to promote the action of IGF-I, pathological abnormalities related to aging, such as neuro-degenerative disorders, may also obtain satisfactory benefit from its use.

Illustrative, non-limiting examples of formulations according to the invention are reported hereinbelow.

| | | | |
|---|---|---|---|
| 1) | Acetyl L-carnitine | mg | 500 |
| | Hydroxytyrosol | mg | 5 |
| 2) | Carnitine mixture | mg | 500 |
| | (acetyl-L-carnitine, propionyl-L-carnitine, | | |
| | isovaleryl-L-carnitine in various | | |
| | weight amounts) | | |
| | Hydroxytyrosol | mg | 5 |
| 3) | Acetyl L-carnitine | mg | 250 |
| | Hydroxytyrosol | mg | 4 |
| 4) | Carnitine mixture | mg | 250 |
| | (acetyl-L-carnitine, propionyl-L-carnitine, | | |
| | isovaleryl-L-carnitine in various | | |
| | weight amounts) | | |
| | Hydroxytyrosol | mg | 4 |
| 5) | Acetyl L-carnitine | mg | 1 |
| | Hydroxytyrosol | mg | 100 |
| 6) | Acetyl L-carnitine | mg | 250 |
| | Hydroxytyrosol | mg | 5 |
| | Selenium methionine | μg | 50 |
| | Zinc glycinate | mg | 10 |
| | Magnesium stereate | mg | 20 |
| | Taurine | mg | 50 |
| | Vitamin E | mg | 10 |
| | Coenzyme Q10 | mg | 10 |
| | β-carotene | mg | 10 |
| | Vitamin C | mg | 30 |

What is claimed is:

1. A composition comprising
(a) acetyl-L-carnitine or a pharmacologically acceptable salt thereof; and
(b) a mixture of polyphenols containing hydroxytyrosol in an effective weight ratio.

2. The composition of claim 1, wherein ingredient (a) further comprises a carnitine selected from the group consisting of L-carnitine, propionyl L-carnitine, valeryl-L-carnitine, isovaleryl-L-carnitines, and their pharmacologically acceptable salts, or mixtures thereof.

3. The composition of claim 1 wherein the weight ratio (a):(b) is from 100:1 to 1:10.

4. The composition of claim 1 wherein a pharmacologically acceptable salt of acetyl-L-carnitine is selected from the group consisting of chloride, bromide, iodide, aspartate, acid aspartate, citrate, acid citrate, tartrate, phosphate, acid phosphate, fumarate, acid fumarate, glycerophosphate, glucose phosphate, lactate, maleate, acid maleate, orotate, acid oxalate, sulphate, acid sulphate, trichloroacetate, trifluoroacetate and methane sulfonate.

5. The composition of claim 2 wherein the carnitine selected is the pharmacologically acceptable salt of L-carnitine or alkanoyl-L-carnitine selected from the group consisting of chloride, bromide, iodide, aspartate, acid aspartate, citrate, acid citrate, tartrate, phosphate, acid phosphate, fumarate, acid fumarate, glycerophosphate, glucose phosphate, lactate, maleate, acid maleate, orotate, acid oxalate, sulphate, acid sulphate, trichloroacetate, trifluoroacetate and methane sulphonate.

6. The composition of claim 1 further comprising vitamins, coenzymes, mineral substances or other antioxidants.

7. The composition of claim 1 in an orally administrable form as a dietary supplement.

8. The composition of claim 1 in an orally, parenterally, rectally or transdermally administrable form as a medicament.

9. The composition of claim 7 in solid, semi-solid or liquid form.

10. The composition of claim 9 in the form of tablets, lozenges, pills, capsules, granulates or syrups.

11. The composition of claim 10 in the form of tablets, lozenges, pills, capsules, granulates, syrups, injection or drops.

12. A method of preventing tissue damage due to free radicals generated by environmental pollution, of preventing brain or myocardial lesions induced by free radicals following cerebral or myocardial ischemia and attendant reperfusion, of preventing diabetic or toxic neuropathies, or metabolic disorders in glucose utilization, said method comprising administering to a subject a composition comprising:
(a) acetyl-L-carnitine or a pharmacologically acceptable salt thereof and a carnitine selected from the group consisting of L-carnitine, propionyl-L-carnitine, valeryl-L-carnitine, isovaleryl-L-carnitine or their pharmacologically acceptable salts or mixtures thereof; and
(b) a mixture of polyphenols containing hydroxytyrosol.

13. A method of treating a disease caused by free radicals due to environmental pollution; brain or myocardial lesions induced by free radicals following cerebral or myocardial ischemia and attendant reperfusion; atherosclerotic lesions and tissue proliferative processes; diabetic or toxic neuropathies; and metabolic disorders of glucose utilization, said method comprising administering to a subject a composition comprising
(a) acetyl-L-carnitine or a pharmacologically acceptable salt thereof and optionally a carnitine selected from the group consisting of L-carnitine, propionyl L-carnitine, valeryl L-carnitine, isovaleryl L-carnitine or their pharmacologically acceptable salts or mixtures thereof; and
(b) a mixture of polyphenols containing hydroxytyrosol.

14. The method of claim 12 wherein the weight ratio (a):(b) is from 100:1 to 1:10.

15. The method of claim 13 wherein the weight ratio (a):(b) is from 100:1 to 1:10.
